(19) United States
(12) Patent Application Publication
(10) Pub. No.: US 2010/0179561 A1
(43) Pub. Date: Jul. 15, 2010

(54) TOOL FOR RETRACTING A TINE ELEMENT OF A MEDICAL LEAD

(75) Inventors: Craig S. Pilarski, Ham Lake, MN (US); Thomas I. Miller, Plymouth, MN (US); Christopher J. Poletto, North Oaks, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/683,757

(22) Filed: Jan. 7, 2010

Related U.S. Application Data
(60) Provisional application No. 61/143,430, filed on Jan. 9, 2009.

(51) Int. Cl. A61B 17/00; A61N 1/05

(52) U.S. Cl. 606/129; 607/116

(57) ABSTRACT

A tool for retracting a tine of a lead includes a body extending from a proximal end to a distal end. The body defines
(i) a lumen configured to receive the lead and
(ii) a longitudinal slit in communication with the lumen.
   The longitudinal slit is configured to slidably receive the tine when the tine is deployed such that the deployed tine extends though the longitudinal slit. The tool and slit are configured such that axial rotation or longitudinal retraction of the tool causes the tine to retract into the lumen.

### RELATED APPLICATION

This application claims priority to U.S. Provisional Application No. 61/143,430, filed Jan. 9, 2009. U.S. Provisional Application No. 61/143,430 is hereby incorporated herein by reference in its entirety.

### FIELD

This application relates to medical devices, more particularly implantable leads having tine retention elements and tools for retracting deployed tine retention elements.

### BACKGROUND

A variety of implantable medical devices have been proven to be effective for treatment of a variety of diseases. Many of such devices, such as cardiac pacemakers, defibrillators, spinal cord or deep brain stimulators, gastric stimulators, and the like employ accessory medical leads to deliver electrical signals from signal generating device to tissue of a patient at a location removed from the signal generating device. Typically the lead is tunneled from a subcutaneous region of the patient in which the signal generating device is implanted to a target tissue location. It is often important that the lead, or portions thereof, does not shift or move once implanted to ensure that a therapeutic signal continues to be delivered to the target tissue. One mechanism for retaining the implanted position of a lead or portion thereof is the use of tines. The tines are typically attached to various locations of the lead and are deployed once the lead is properly positioned in the patient. Most often, tines prevent retrograde movement of the lead. Once the tines are deployed, it can be difficult to change the position of the lead.

### BRIEF SUMMARY

In various embodiments, the present disclosure relates to systems, devices and methods for retracting deployed tines. Once the tines are retracted, a lead may be more readily withdrawn or repositioned.

In an embodiment, a tool for retracting a tine of a lead includes a body extending from a proximal end to a distal end. The body defines (i) a lumen configured to receive the lead and (ii) a longitudinal slit in communication with the lumen. The longitudinal slit is configured to slidably receive the tine when the tine is deployed such that the deployed tine extends though the longitudinal slit. The tool and slit are configured such that axial rotation or longitudinal retraction of the tool causes the tine to retract into the lumen.

### BRIEF DESCRIPTION OF THE DRAWINGS

FIG. 1 is a schematic exploded view of a representative implantable electrical signal therapy system.

FIG. 2 is a schematic perspective view of a representative lead.

FIG. 3 is a schematic perspective view of a representative lead.

FIG. 4 is a diagrammatic representation of a representative spinal cord stimulation (SCS) system implanted in a patient.

FIG. 5 is a diagrammatic representation of a representative bifurcated lead implanted in a patient.

FIG. 6 is a schematic perspective view of a lead with tines.

FIG. 7 is a schematic perspective view of a representative tine element.

FIGS. 8A-B are schematic perspective views of a lead received by a tine retraction tool.

FIG. 9 is a schematic radial cross section of a lead disposed in a tine retraction tool.

FIGS. 10-11 are schematic perspective views of tine retraction tools.

FIGS. 12A-B are schematic side views of a lead with a deployed (12A) and retracted (12B) tine.

FIG. 13A is a schematic side view of a lead having two radially spaced apart tines.

FIG. 13B is a schematic radial cross section of a tine retraction tool having two radially spaced apart longitudinal slits.

FIG. 14A is a schematic radial cross section of a tine retraction tool having three radially spaced apart longitudinal slits.

FIG. 14B is a schematic side view of a lead having three radially spaced apart tines.

FIGS. 15A-D are schematic radial cross sections of a lead disposed in a lumen of a tine retraction tool, where the tool is being used to deploy the tine.

FIG. 16A is a schematic perspective view of a lead, tool, and outer jacket.

FIGS. 16B-C, FIGS. 17A-B, and FIGS. 18A-B are schematic cross sections of various embodiments of leads, tools, and outer jackets.

FIGS. 19A-B are schematic cross sections of a tool and pushing member.

FIGS. 20A-D are schematic perspective views of a tine retraction tool and a lead.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope of spirit of the present invention. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

In various embodiments, the present disclosure relates to systems, devices and methods related to retracting deployed tines. The tines are associated; e.g., affixed or integrally formed, with an implantable medical lead. Once deployed, the tines resist withdrawal of the lead. Tools for retracting the lead are described herein. Once the tines are retracted, the lead may be withdrawn, moved to a more desirable location, or the like. The lead may be associated with an active implantable medical device, such as a hearing implant; a cochlear implant; a sensing or monitoring device; a signal generator such as a cardiac pacemaker or defibrillator, a neurostimulator (such as a spinal cord stimulator, a brain or deep brain stimulator, a peripheral nerve stimulator, a vagal nerve stimulator, an occipital nerve stimulator, a subcutaneous stimulator, etc.), a gastric stimulator; or the like.

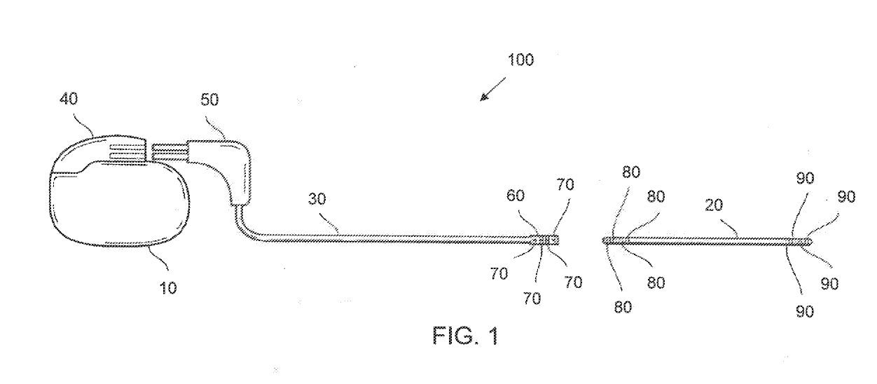

Referring to FIG. 1, a schematic exploded view of a representative implantable active electrical system 100 is shown. In the system shown in FIG. 1, implantable active electrical device 10 comprises a connector header 40 configured to receive connector 50 at the proximal end of lead extension 30. Of course, it will be understood that device 10 need not have a separate header 40 to receive extension 30. The distal end of extension 30 comprises a connector 60 configured to receive proximal end of lead 20. Connector 60 comprises internal electrical contacts 70 configured to electrically couple extension 30 to lead 20 via electrical contacts 80 disposed on the proximal end portion of lead 20. Electrodes 90 are disposed on distal end portion of lead 20 and are electrically coupled to electrical contacts 80, typically through conductors (not shown). Lead 20 may include any number of electrodes 90, e.g. one, two, three, four, five, six, seven, eight, sixteen, thirty-two, or sixty-four. Typically, each electrode 90 is electrically coupled to a discrete electrical contact 80. While not shown, it will be understood that lead 20 may be directly coupled to active implantable medical device 10 without use of extension 30 or adaptor in some systems 100.

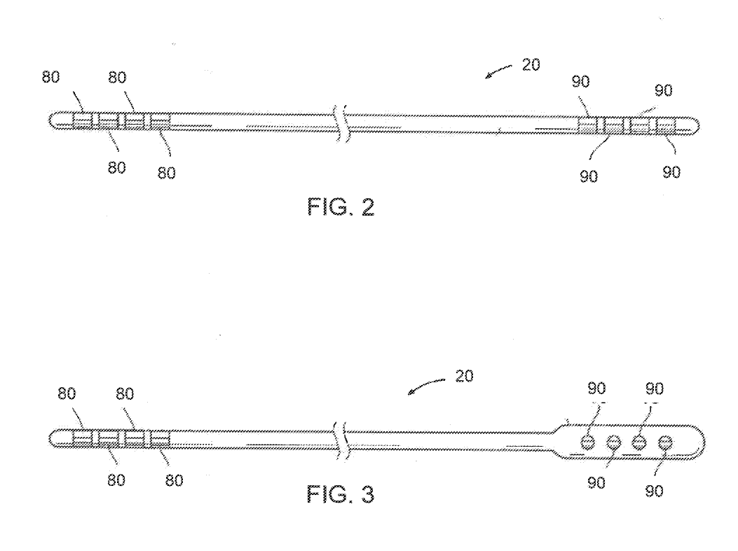

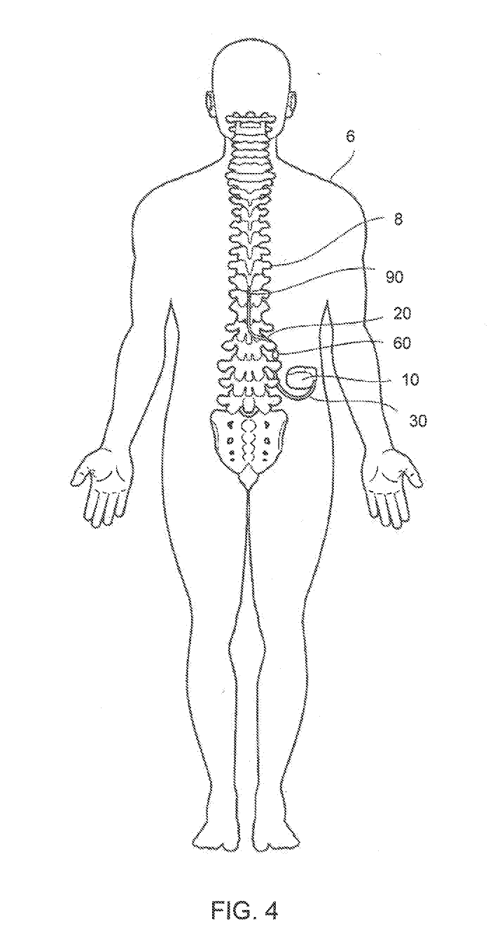

FIGS. 2 and 3 are schematic perspective views of representative leads 20. Leads 20, as shown in FIGS. 2 and 3, contain four exposed electrical contacts 80 and four electrodes 90. The lead 20 shown in FIG. 2 is cylindrical throughout. Examples of such leads include percutaneous leads. The lead 20 shown in FIG. 3 contains a paddle-shaped distal portion. Such leads are often referred to as surgical leads. While only two types of lead configurations are shown, it will be understood that any lead configuration may be employed in accordance with the teachings provided herein.

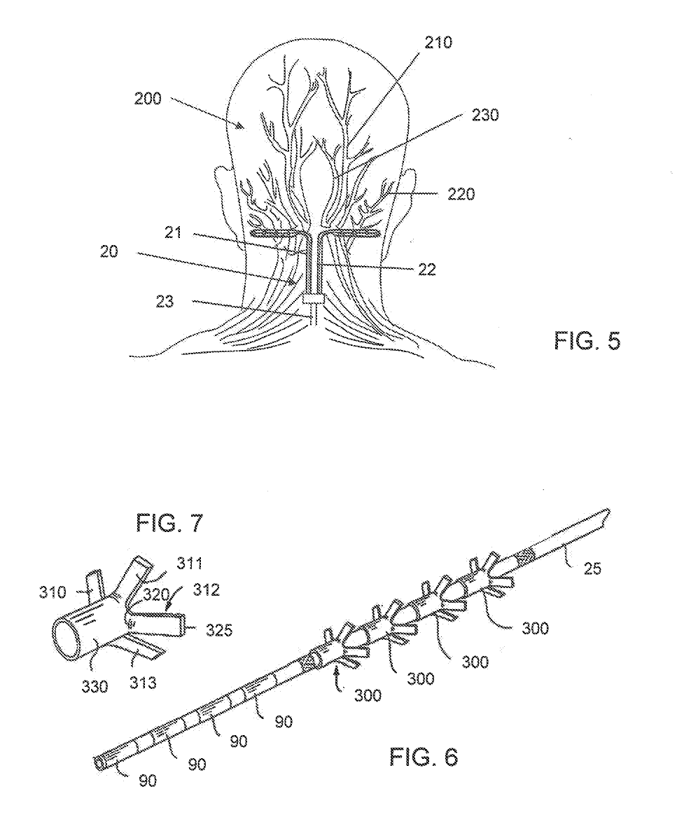

By way of example and referring to FIG. 4, a spinal cord stimulation (SCS) system is shown implanted in a patient 6. For SCS, an implantable pulse generator (IPG) 10 is typically placed in the abdominal region of patient 6 and lead 20 is placed at a desired location along spinal cord 8. Such a system, or any system including an IPG 10 as described herein, may also include a programmer (not shown), such as a physician programmer or a patient programmer. IPG 10 is capable of generating electrical signals that may be applied to tissue of patient 6 via electrodes 90 for therapeutic or diagnostic purposes. IPG 10 contains a power source and electronics for sending electrical signals to the spinal cord 8 via electrodes 90 to provide a desired therapeutic effect. It will be appreciated that other systems employing active electrical devices and therapeutic uses thereof are contemplated.

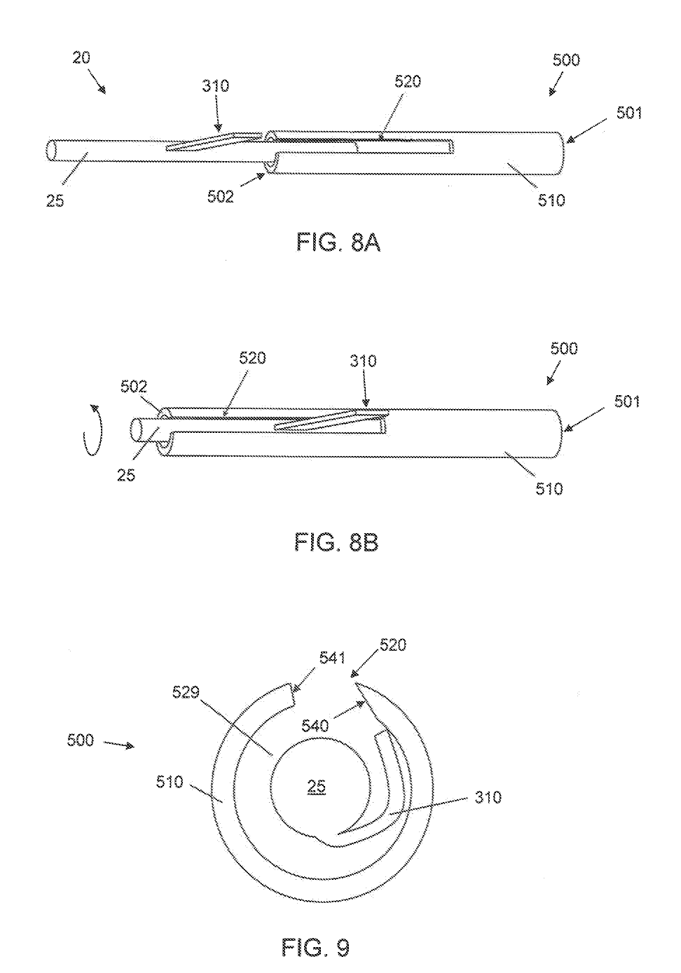

By way of further example and referring to FIG. 5, lead 20 is shown implanted in a patient to provide bilateral therapy to left and right occipital nerves 200. Lead 20 is bifurcated and includes first 21 and second 22 branches forming from a proximal stem portion 23. Of course, two separate leads or lead extensions may be employed for providing electrical signals to occipital nerves 200. As used herein, occipital nerve 200 includes the greater occipital nerve 210, the lesser occipital nerve 220 and the third occipital nerve 230. The greater and lesser occipital nerves are spinal nerves arising between the second and third cervical vertebrae (not shown). The third occipital nerve arises between the third and fourth cervical vertebrae. The portion of the occipital nerve 200 to which an electrical signal is to be applied may vary depending on the disease to be treated and associated symptoms or the stimulation parameters to be applied. In various embodiments, the lead distal portions that contain electrodes are placed to allow bilateral application of electrical signals to the occipital nerve 200 at a level of about C1 to about C2 or at a level in proximity to the base of the skull. The position of the electrode(s) may vary. In various embodiments, one or more electrodes are placed between about 1 cm and about 8 cm from the midline to effectively provide an electrical signal to the occipital nerve 200.

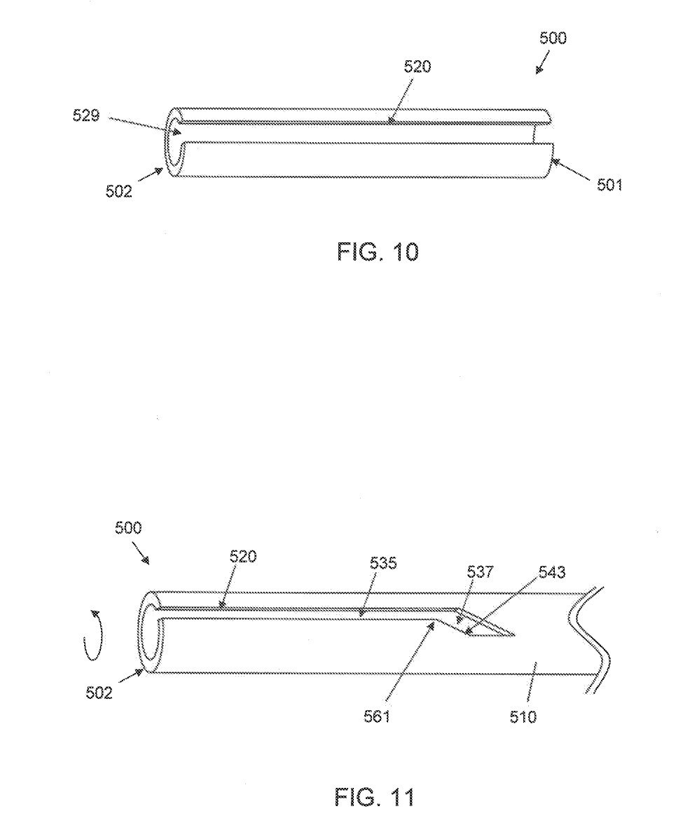

Referring to FIG. 6, a lead 20 including tine elements 300 is shown. The tine elements 300 may be associated with the lead 20 in any suitable manner. For example, one or more tine elements 300 may be disposed about the lead body 25 or may be integrally formed with lead body 25. In the depicted embodiment, the tine elements 300 are disposed in proximity to the distal end of the lead 20 proximal to the electrodes 90. However, the tine elements 90 may be located at any suitable and desirable location along the lead 20. If a tine element 300 is disposed about the lead body 25, the tine element 300 may be fixed relative to the lead body 25 via any suitable mechanism, such as crimping, adhesive, fastener, or the like. A tine element 300 may have any number of tines.

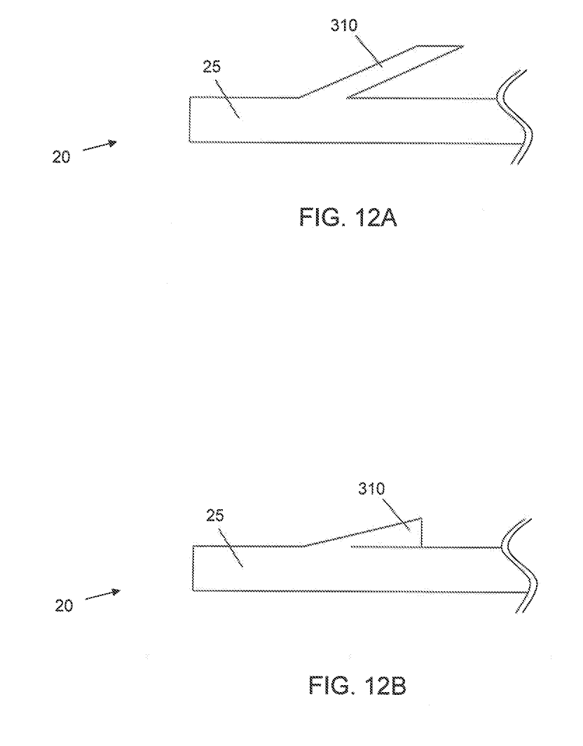

For example and referring to FIG. 7, a tine element having four tines 310, 311, 312, 313 is shown. The tine element depicted in FIG. 7 includes'a mounting band 330. The mounting band 330 is configured to encircle a lead body with the tines 310, 311, 312, 313 extending from respective attached tine ends or roots disposed apart from one another around the tine mounting band 330. The tines 310, 311, 312, 313 preferably have a thickness that enables folding of the tines against the body of the lead about which they are disposed. In the depicted embodiment, the tines 310, 311, 312, 313 extend radially outward and proximally at about 45 degrees to the axis of the lead body and mounting band 330 in their relaxed and deployed state. Of course the tines may extend outwardly at nearly any suitable degree to the axis of the lead body or mounting band, if present.

It will be understood that nearly any suitable tine or tine element may be employed with the teachings presented herein. A tine and components of a tine element may be made of any suitable material. For example, the tines or components of the tine elements may be formed from a bio-compatible plastic, such as medical grade silicone rubber or polyurethane, from a superelastic alloy material, or the like.

While discussed above as separate components, lead and lead extension will be used herein below interchangeably, unless clearly indicated to the contrary.

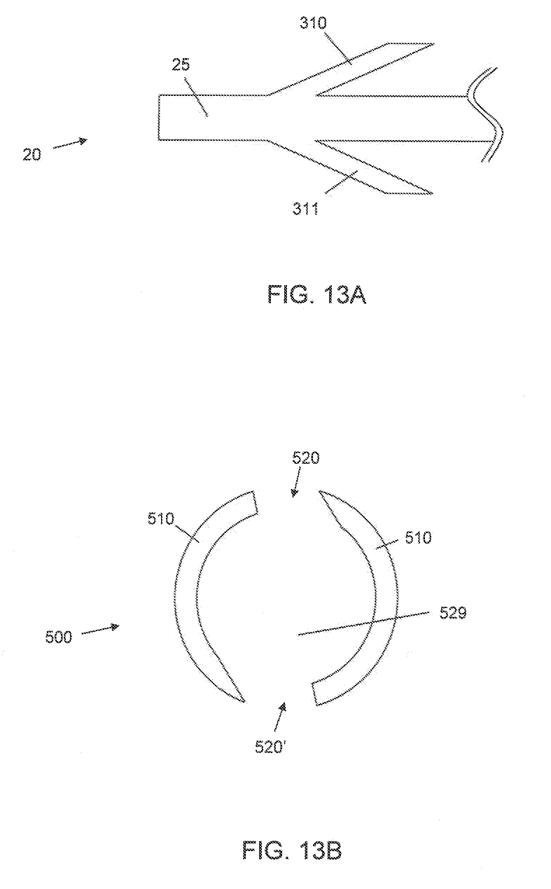

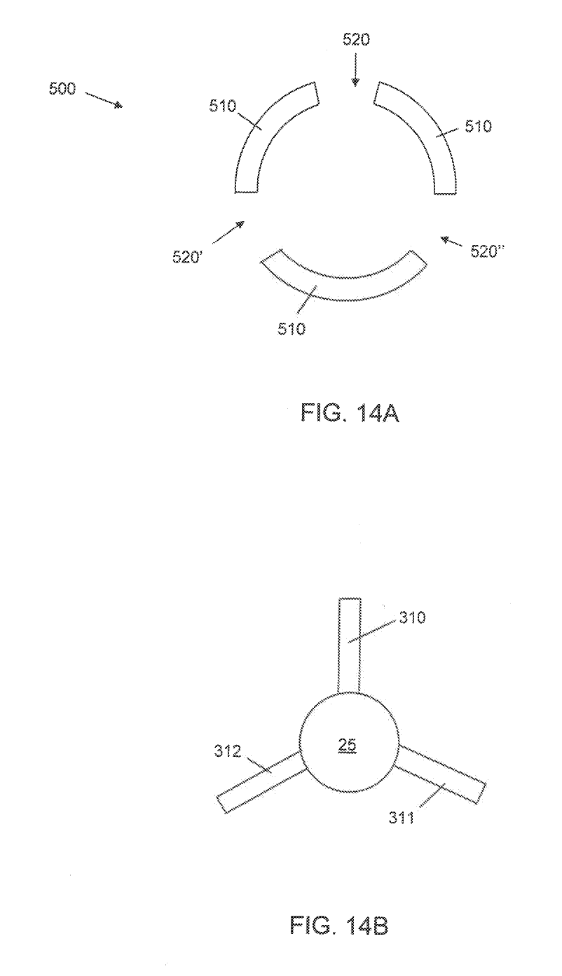

Referring now to FIGS. 8A-B, a schematic perspective side view of a lead 20 and tine retraction tool 500 are shown. The depicted tine retraction tool 500 includes a tool body 510 that extends from a tool proximal end 501 to a tool distal end 502. The tool body 510 defines a lumen 529 (see FIG. 9) and a longitudinal slit 520 in communication with the lumen. The lumen extends through the distal end 502 of the tool and is configured to slidably receive the lead 20, or a portion thereof. The longitudinal slit 520 is configured to receive a tine 310 of the lead 20 when the tine 310 is deployed such that the deployed tine 310 extends through the longitudinal slit 520. In the depicted embodiment, the tine 310 is shown integrally formed with the body 25 of the lead 20. However, the tine 310 may be a part of a tine element disposed about the lead body. As indicated by the arrow in FIG. 8B, the tool 500 may be rotated axially relative to the lead 20 when the tine 310 is received by the slit 520. The tool 500 is configured such that axial rotation caused the tine 310 to retract into the lumen 529 of the tool 500 (see, e.g.; the schematic radial cross-section depicted in FIG. 9).

As further shown in the embodiment depicted in FIG. 9, first 540 and second 541 sidewalls of the tool body 510 form the longitudinal slit 520. The first side wall 540 forms a ramp to facilitate retraction of the tine 310 into the lumen 529 when the tool 500 is axially rotated relative to the lead body 25. In the depicted embodiment, the first sidewall 540 forms an acute angle with the outer surface of the body 510 of the tool 500 and forms an obtuse angle with the inner surface of the body 510 at the intersection with the side wall 540. It will be understood that both sidewalls 540, 541 may form such a ramp. It will be further understood that any suitable mechanism may be employed to facilitate retraction of the tine 310; e.g., the first sidewall 540 may be rounded, or the like.

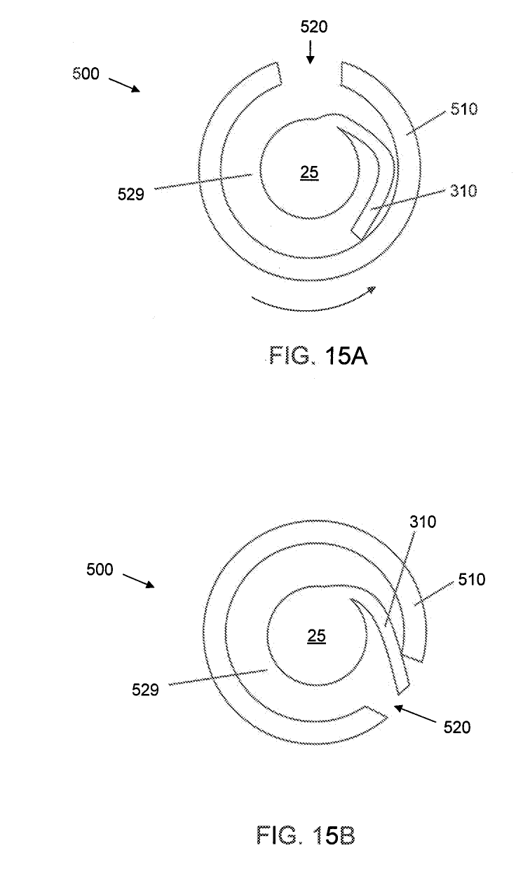

Referring to FIG. 10, a tine retraction tool 500 having a longitudinal slit 520 extending from the proximal 501 to distal 502 end of the tool 500 is shown. Of course, the slit 520 may extend proximally any suitable distance. In the embodiment depicted in FIG. 9, the tool lumen 529 also extends from the proximal 501 to the distal 502 end. With the lumen 529 extending through the tool body 510, a proximal end of a lead received by the lumen 529 may extend beyond the proximal end 501 of the tool 500, allowing the tool to have a length less than that of the lead that it is configured to receive.

In the embodiments depicted in FIGS. 8 and 10, the slit 520 has a substantially uniform width along its length (e.g., the width does not vary more than 5% at any point along its length). However, it will be understood that the width may vary along the length of the slit 520. The slit 520 depicted in FIGS. 8 and 10 is substantially linear along its length (e.g., does not vary more than 5%, based on length, from linear). However, it will be understood that the slit 520 may curve along its length.

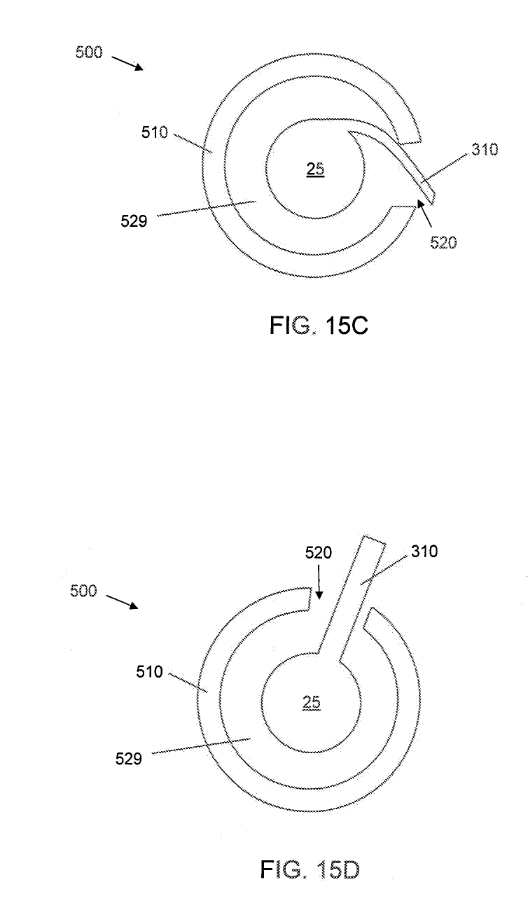

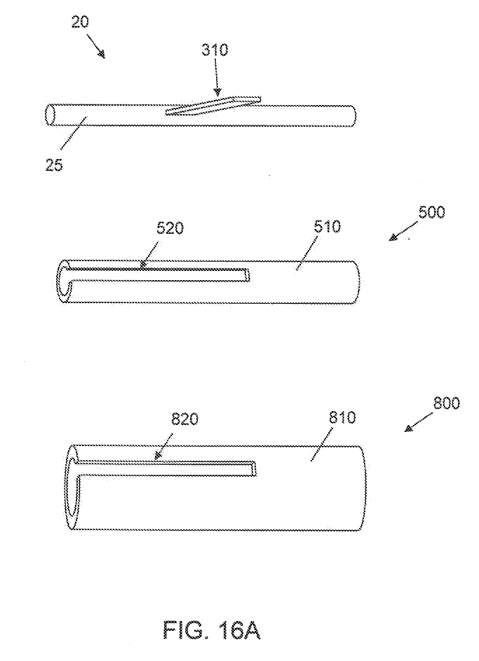

Referring now to FIG. 11, a perspective view of an embodiment of a tine retraction tool 500 is shown. In the depicted embodiment, the slit 520 forms a first substantially linear section 535 that extends from the distal end 502 to a first location 561 of the tool body 510. The slit 520 further includes a second portion 537 extending proximally from the first section 535 at an obtuse angle. The face of the distal side wall 543 of the second portion 537 of the slit 520 may include a ramp, be rounded, or the like to facilitate retraction of at tine as the tool 500 is axially rotated relative to a lead including a tine. If the tool 500 is advanced over the lead such that the tine engages the distal side wall 543 of the second portion 537 of the slit 520 as the tool 500 is rotated, the obtuse angular arrangement of the sidewall 543 may encourage the tine to deflect longitudinally back along the lead body as opposed to radially. For example and with reference to FIGS. 12A-B, schematic side views of a lead 20 having a deployed tine 310 (12A) and a lead 20 having a tine 310 deflected longitudinally along the lead body 25 (12B) are shown.

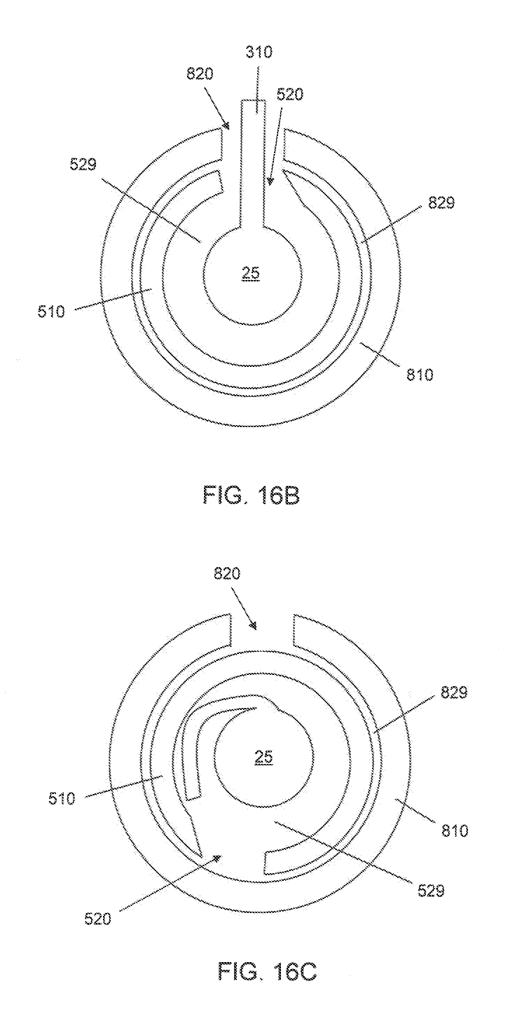

Referring now to FIGS. 13A-B and 14A-B, tine retraction tool body 510 may define more than one longitudinal slit 520, 520′, 520″ in communication with lumen 529. In such cases, the slits 520, 520′, 520″ may extend from the distal end of the tool 500 to a location distal the proximal end. The number of longitudinal slits 520, 520′, 520″ in the tool 500 may be matched to the number of radially spaced apart tines 310, 311, 312 of a lead 20. Of course, if two or more tines are longitudinally aligned along the length of a lead, a single longitudinal slit may be sufficient for retracting the two or more tines.

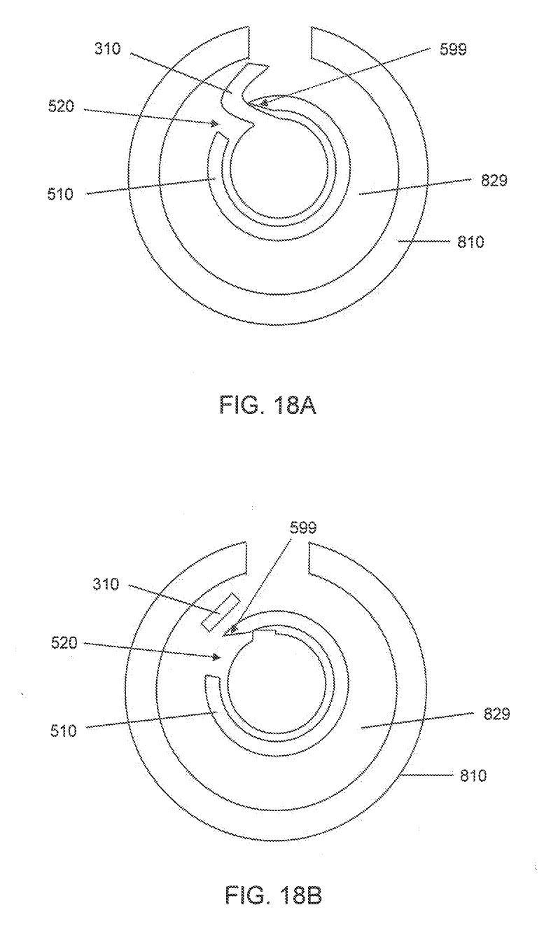

With reference now to FIGS. 15A-D, in various embodiments a tine retraction tool 500 can be employed to deploy a retracted tine 310. In the depicted embodiment, the tool 500 is axially rotated relative to the body 25 of the lead in the direction of the arrow shown in FIG. 15A. As the tool 500 is rotated such that a distal portion of the tine 310 is aligned with the slit 520, the distal portion of the tine 310 extends through the slit 520 due to the resilient nature of the tine 310 (see FIG. 15B). Further axial rotation of the tool 500 allows more of the tine 310 to extend from the slit 520 (FIG. 15C) until the tine is fully deployed (FIG. 15D). In the depicted embodiment, the tine 310 is retracted radially within the lumen 529 of the tool 500. If the tine were longitudinally retracted, the tine would spring to its deployed, relaxed state when the slit is aligned with the tine. Upon deployment, the tool 500 may be removed with the lead being anchored in a patient via the deployed tine 310.

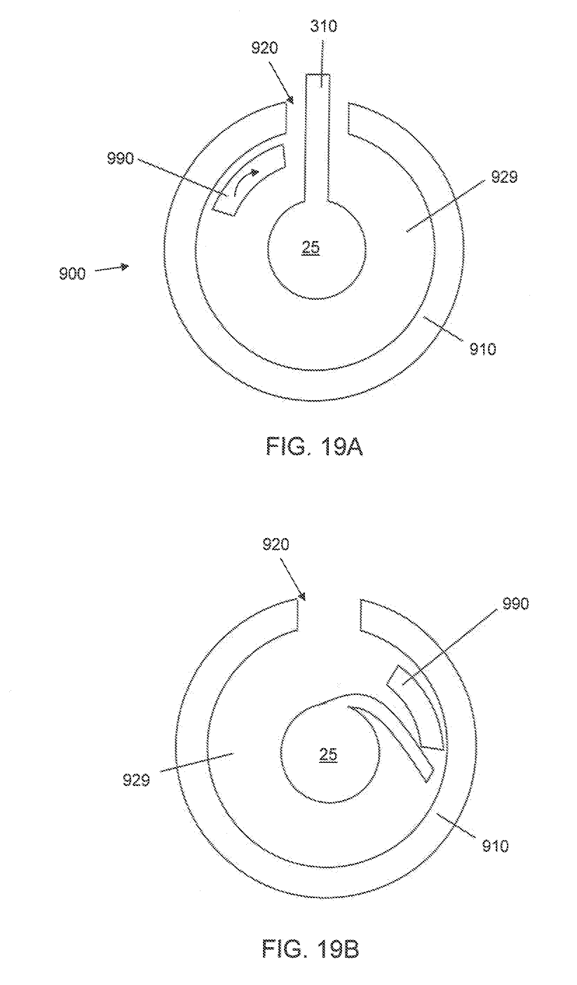

A tool as described herein may be made of any suitable material. Preferably, the material is biocompatible and sufficiently rigid to cause deflection and retraction of a tine of a lead. Examples of suitable materials include polymers such as polysulfone, polycarbonate, or the like; metallic materials such as stainless steel, titanium, or the like. The tools may be molded or formed by any other suitable process. Slits may be molded into the tool, or may be cut or otherwise formed from a cylindrical tool precursor. Referring now to FIG. 16A-C, a system including a tool 500, an outer jacket 800, and a lead 20 is shown. The tool 500, as described above, includes a body member 510 forming a longitudinal slit 520. The outer jacket 800 includes a body member 810 forming a longitudinal slit 820. The longitudinal slit 820 extends from the distal end of the body 810 to a location proximal the distal end. Of course the slit 820 may extend to the proximal end of the body 810 (e.g. as shown in FIG. 10 with tool 500). The slit 820 is in communication with lumen 829 formed by body 810. The lumen 829 is configured to slidably receive the tool 500. The tool 500 is configured to be axially rotatable within the lumen 829 of the jacket 800. The slit 520 of the tool 500, or a portion thereof, is alignable with the slit 820 of the jacket 800. When aligned, a tine 310 of a lead received by the lumen 529 of the tool 500 may extend through both slits 520, 820 (see FIG. 16B). When the tine 310 extends through both slits 520, 820, the tool 500 may be rotated within the lumen 829 of the jacket 800 to cause the tine 310 to be retracted into the lumen 529 of the tool 500 (see FIG. 16C). In an alternative embodiment and with reference to FIGS. 17A-B, the tool may be axially rotated in the lumen 829 of the jacket such that the tine 310 is retracted into the lumen 829 of the jacket. In an alternative embodiment and with reference to FIGS. 18A-B, the sidewall 599 of tool body 510 forming the longitudinal slit 520 forms or includes a cutting edge. Sufficient axial rotation of the tool within the lumen 829 of the jacket causes the cutting edge to advance relative to a tine 310 of a lead received by the tool to cut the tine 310.

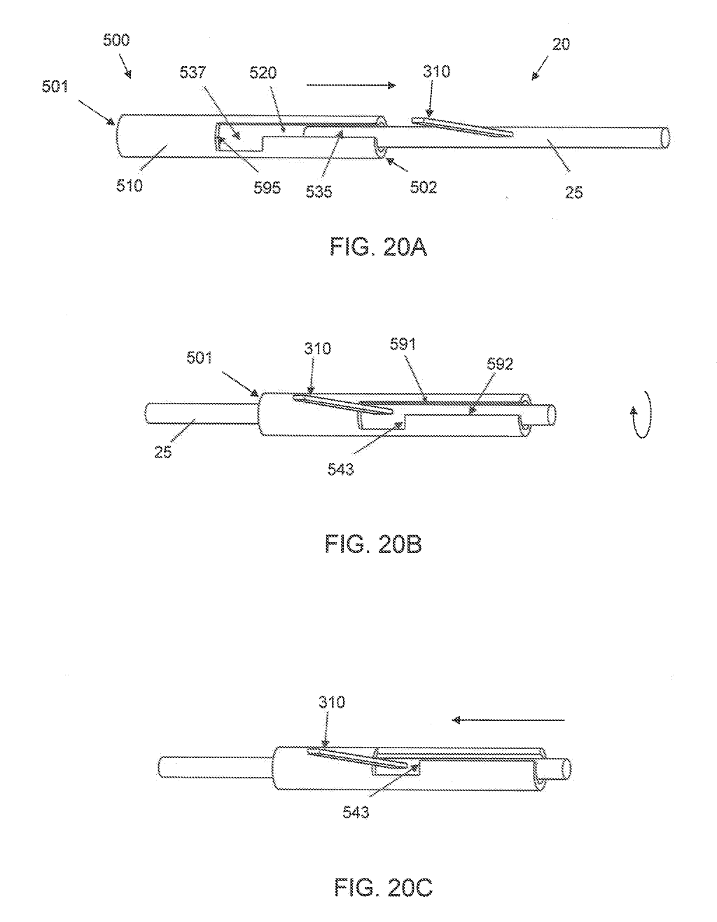

Referring now to FIGS. 19A-B, a schematic cross-section of a tool 900 is shown. Perspective views of the tool 900 may be similar to, for example, the tools depicted in

FIG. 8, 10, or 11. The tool 900 has a body 810 forming a lumen 929 and a longitudinal slit 920. The lumen 929 is configured to slidably receive a body 25 of a lead. The longitudinal slit 920 of the tool 900 is configured to slidably receive a tine 310 of the lead. A pushing member 990 may be disposed within the lumen 929 of the tool 900. The pushing member 990 may be moved relative to the tool body 910 and slit 920 to engage the tine 310 and cause the tine 310 to retract into the lumen 929 of the tool 900. To accomplish such tine retraction, the pushing member 990 may be advanced from one side of the longitudinal slit to beyond the other side of the longitudinal slit.

It will be understood that tools with pushing members as described with regard to FIGS. 19A-B may be generally take the form of the systems described with regard to

FIGS. 16-18, where the pushing member 990 replaces the tool 500. For example, the pushing member 990 may include a cutting edge as described with regard to FIGS. 18A-B.

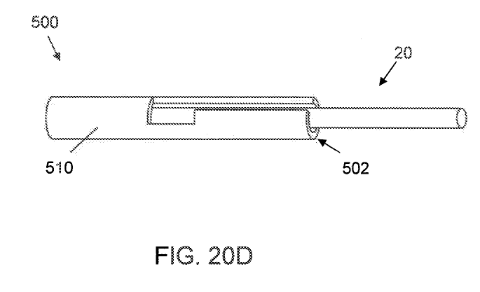

Referring now to FIGS. 20A-D, an alternative embodiment of a tine retraction tool 500 and method for retracting a tine 310 of a lead 20 are shown. The tool 500 includes a body 510 having distal 502 and proximal 501 ends. The body forms a lumen and a slit 520 in communication with the lumen. The slit 520 includes first 535 and second 537 portions. The first portion 535 extends from the distal end 502 to a first location 561 proximal the distal end. The second portion 537 is in communication with the first portion 535 and extends generally orthogonally from the first portion. The second portion 537 has a width that is greater than the width of the first portion 535. In the depicted embodiment, the first 535 and second 537 portion of the longitudinal slit 520 share a common edge 591. A distal edge 543 forms a portion of the second portion 537 of the slit 520. The distal edge 543 may form a ramp, be curved, or the like to facilitate retraction of the tine 310, as described in more detail below. The distal edge 543 extends generally orthogonally from an edge 592 of the first portion 535 of the slit 520. Edge 592 of the first portion 535 is generally parallel to and opposed from the common edge 591.

In the embodiment depicted in FIGS. 20A-D, longitudinal slit 520 is configured to slidably receive a deployed tine 310 of lead 20. Lead 20 may be received by the lumen of the tool 500 such that slit 520 is longitudinally aligned with tine 310 (see FIG. 20A). Tine 310 may be slid within slit 520 as tool 500 is slid distally (in direction of arrow in FIG. 20A) relative to lead 20. When tine 320 engages proximal face 595 of slit 520, the tool 500 may be axially rotated (in direction of arrow in FIG. 20B) relative to lead body 25 to cause the tine 310 to occupy the second portion 537 of the slit. Tactile feedback due to tine 310 engaging the edge of the second portion 537 opposed to the common edge 591 may be used to determine whether tool 500 has been sufficiently rotated. The tool 500 may be moved proximally (in direction of arrow in FIG. 20C) relative to the lead 20 to cause the tine 310 to engage the distal face 543 of the second portion 537 of the slit 520. Further proximal movement of the tool 500 relative to the lead 20 will cause retraction of the tine 310 into the lumen of the tool (see FIG. 20D). The tine 310 in the depicted embodiment would fold back proximally along the lead body. The lead 20 may then be withdrawn from the patient in whom it is implanted or repositioned as desired. The tine 310 may then be redeployed by axially rotating the tool 500 until the slit 520 and the tine 310 are aligned or by moving the tool 500 distally relative to the lead 220 to allow the tine 320 to deploy through the second portion 537 of the slit 520.

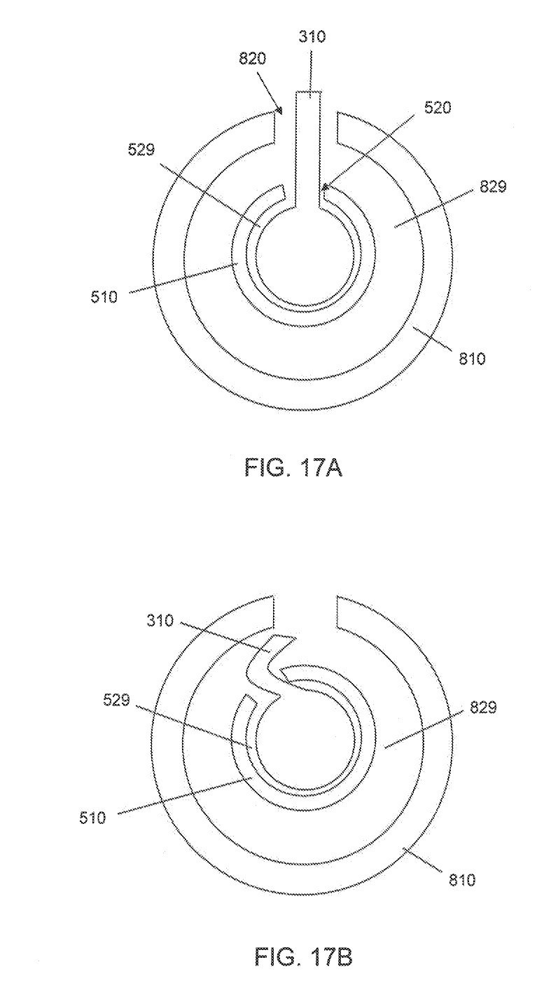

While the tools and jackets depicted herein have been shown as being generally cylindrical, it will be understood that, in various embodiments, it may be desirable for such tools and jackets to be formed into other shapes. For example, if a tine is disposed on a portion of a paddle of a paddle lead, it may be desirable for a tool or jacket to have a rectangular or oval cross-sectional shape. Such alternative shapes are contemplated herein. It will be understood that various aspects or components of the various drawings and embodiments described herein may be used interchangeably. For example, a tool having a slit with first and second sections where the second section extends generally orthogonally from the first section (e.g., as shown in FIGS. 20A-D) may include two or more of slits (e.g., as shown in FIG. 13B or FIG. 14A).

Thus, embodiments of the TOOL FOR RETRACTING A TINE ELEMENT OF A MEDICAL LEAD are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A tool for retracting a tine of a lead, comprising:
a body extending from a proximal end to a distal end, the body defining
(i) a lumen configured to receive the lead and
(ii) a longitudinal slit in communication with the lumen,
wherein the longitudinal slit is configured to slidably receive the tine when the tine is deployed such that the deployed tine extends though the longitudinal slit, and
wherein the tool is configured such that axial rotation of the tool causes the tine to retract into the lumen.

2. A tool according to claim 1, wherein the longitudinal slit extends through the distal end of the tool body.

3. A tool according to claim 1, wherein the longitudinal slit extends through the proximal end of the tool body.

4. A tool according to claim 1, wherein first and second sidewalls of the tool body form the longitudinal slit and wherein the first sidewall forms a ramp to facilitate retraction of the tine into the lumen when the tool is axially rotated.

5. A tool according to claim 1, wherein the slit has a uniform width along its length.

6. A tool according to claim 1, wherein the slit is linear.

7. A tool according to claim 1, wherein the slit forms a first linear section between a distal end and a first location of the tool body, the first location being proximal the distal end, and wherein the slit forms a second linear section extending proximally from the first section at an obtuse angle.

8. A tool according to claim 7, wherein the second section of the slit is formed in part by a distal side wall, wherein the distal side wall comprises a ramp to facilitate retraction of the tine into the lumen when the tool is axially rotated.

9. A system comprising:
an implantable medical lead having
(i) a lead body extending from a proximal end to a distal end and
(ii) a tine extending from the lead body; and
a tine retraction tool having a body extending from a proximal end to a distal end, the tool body defining
(i) a lumen configured to receive the lead body and
(ii) a longitudinal slit in communication with the lumen,
wherein the longitudinal slit is configured to slidably receive the tine when the tine is deployed such that the deployed tine extends though the longitudinal slit, and
wherein the tool is configured such that axial rotation of the tool causes the tine to retract into the lumen.

10. A system according to claim 9, wherein the longitudinal slit extends through the distal end of the tool body.

11. A system according to claim 9, wherein the longitudinal slit extends through the proximal end of the tool body.

12. A system according to claim 9,'wherein first and second sidewalls of the tool body form the longitudinal slit and wherein the first sidewall forms a ramp to facilitate retraction of the tine into the lumen when the tool is axially rotated.

13. A system according to claim 9, wherein the slit has a uniform width along its length.

14. A system according to claim 9, wherein the slit is linear.

15. A system of claim 9, wherein the slit forms a first linear section between the distal end and a first location of the tool body, the first location being proximal the distal end, and wherein the slit forms a second linear section extending proximally from the first section at an obtuse angle.

16. A system according to claim 15, wherein the second section of the slit is formed in part by a distal side wall, wherein the distal side wall comprises a ramp to facilitate retraction of the tine into the lumen when the tool is axially rotated.

17. A system according to claim 9, further comprising an outer jacket having a jacket body forming a jacket lumen and a jacket longitudinal slit, wherein the jacket longitudinal slit is in communication with the jacket lumen, wherein the jacket lumen is configured to slidably receive the tine retraction tool, wherein the longitudinal slit of the tool and the jacket longitudinal slit are alignable such that the deployed tine extends through the both the longitudinal slit of the tool and the jacket longitudinal slit, and wherein the tool is configured to be axially rotatable within the jacket to such that axial rotation of the tool causes the tine to retract into the lumen of the tool.

18. A method for retracting a tine of a lead with a tool having a body extending from a proximal end to a distal end, the tool body defining
(i) a lumen configured to receive the lead and
(ii) a longitudinal slit in communication with the lumen, wherein the longitudinal slit is configured to slidably receive the tine when the tine is deployed such that the deployed tine extends though the longitudinal slit, the method comprising:
axially rotating a tine retraction tool to cause the tine to retract into a lumen of the tool.

19. A tine retraction tool comprising:
a body forming a lumen and a longitudinal slit in communication with the lumen, wherein the lumen is configured to receive a lead, and wherein the longitudinal slit is configured to slidably receive a tine deployed on the lead such that the tine extends through the slit; and
a pushing member moveable relative to the body, wherein movement of the pushing member from one side of the longitudinal slit to beyond the other side of the slit causes pushing member to engage the tine and causes the tine to retract into the lumen of the tool.

20. The tool of claim 19, wherein the pushing member comprises a cylindrical body forming a lumen and a longitudinal slit in communication with the lumen,
wherein the lumen of the member is configured to slidably receive the lead,
wherein the slit of the member is configured to slidably receive the lead,
wherein the cylindrical body is slidably disposable in the lumen of the tool body,
wherein the longitudinal slit of the pushing member and the longitudinal slit of the tool are alignable such that the deployed tine extends through the both the longitudinal slit of the tool body and the jacket longitudinal of the pushing member, and
wherein the pushing member is configured to be axially rotatable within the lumen of the tool body such that axial rotation of the pushing member causes the tine to retract into the lumen of the tool body.

21. A tool of claim 19, wherein the pushing member comprises a cutting edge configured to cut the tine as the member is moved from one side of the longitudinal slit to beyond the other side of the slit.

22. A tool for retracting a tine of a lead, comprising:
a body extending from a proximal end to a distal end, the body defining
(i) a lumen configured to receive the lead and
(ii) a longitudinal slit in communication with the lumen; and
wherein the longitudinal slit is configured to slidably receive the tine when the tine is deployed such that the deployed tine extends though the longitudinal slit,
wherein the slit includes
(i) a first section extending from the distal end of the body to a first location proximal the distal end, and
(ii) a second section extending from and in communication with the first section, the second having a width greater than the first section,
wherein a wall of the body forms a distal edge of the second slit such that proximal movement of the body relative to the lead, when the tine is deployed in the second section of the slit, causes the distal edge to engage the tine and further distal movement of the tool relative to the lead causes the tine to retract into the lumen.

23. A tool according to claim 22, wherein the first and second section of the slit share a common edge.

24. A tool according to claim 23, wherein the common edge is linear.

25. A tool according to claim 24, wherein the second section of the slit extends orthogonally from the first section.

26. A method comprising:
introducing proximal portion of a lead into a distal portion of a lumen of a tine retraction tool, aligning a slit of the tine retraction tool with a tine of the lead;
moving the tool distally relative to the lead until the tine extends through the slit;
axially rotating the tool relative to the lead to cause the tine to occupy a second portion of the slit having a distal edge distal the tine;
moving the tool proximally relative to the lead to cause the tine to engage the distal edge and retract into a lumen of the tool.
